(19) United States
(12) Patent Application Publication
(10) Pub. No.: US 2007/0173727 A1
(43) Pub. Date: Jul. 26, 2007

(54) Method and apparatus for isolating the vascular component in digital temerature monitoring

(75) Inventors: Morteza Naghavi, Houston, TX (US); Timothy J. O'Brien, Anoka, MN (US); Craig Jamieson, Houston, TX (US); Mark Christian Johnson, Houston, TX (US)

Correspondence Address: WONG, CABELLO, LUTSCH, RUTHERFORD & BRUCCULERI,;L.L.P., 20333 SH 249, SUITE 600, HOUSTON, TX, 77070, US

(73) Assignee: Endothelix Inc., Houston, TX (US)

(21) Appl. No.: 11/504,225

(22) Filed: Aug. 14, 2006

Related U.S. Application Data
(63) Continuation-in-part of application No. PCT/US05/18437, filed on May 25, 2005.
(60) Provisional application No. 60/707,455, filed on Aug. 12, 2005.
(60) Provisional application No. 60/585,773, filed on Jul. 6, 2004.
(60) Provisional application No. 60/626,006, filed on Nov. 8, 2004.

(51) Int. Cl. A61B 5/02; A61B 5/00

(52) U.S. Cl. 600/483; 600/549; 600/504

(57) ABSTRACT

Methods and apparatus are provided for identifying and minimizing neurovascular contributions to determinations of endothelial function, including by digital temperature monitoring such that an accurate status of vascular reactivity can be obtained. Methods are also provided for identifying individuals having increased sympathetic nervous system activity impacting peripheral vascular function as well as for determining a status of diabetic foot.

### CROSS REFERENCE TO RELATED APPLICATIONS

This application claims priority to under 35 USC §119 to U.S. Provisional Application No. 60/707,455, filed Aug. 12, 2005, the disclosure of which is incorporated by reference in its entirety. This application also a continuation-in-part of, and claims priority under 35 USC §120 to PCT application PCT/US2005/018437, filed May 25, 2005, and published as WO2005/118516, which claims priority under 35 USC §119 to, among others, U.S. Provisional Application No. 60/585,773, filed Jul. 6, 2004 and U.S. Provisional Application No. 60/626,006, filed Nov. 8, 2004, the disclosures of which are incorporated herein by reference in their entirety.

### FIELD OF THE INVENTION

The present invention relates generally to the field of assessing a patient's vascular health including endothelial function by monitoring changes in hemodynamic parameters responsive to the introduction of a vasodilating stimulant.

### BACKGROUND

Endothelial function (EF) is accepted as the most sensitive indicator of vascular function. EF has been labeled a “barometer of cardiovascular risk” (Vita J A, Keaney J F Jr. “Endothelial function: a barometer for cardiovascular risk?” Circulation, 106(6):640-2, 2002) and is well-recognized as the gateway to cardiovascular disease, by which many adverse factors damage the blood vessel. The endothelium has many important functions in maintaining the patency and integrity of the arterial system. The endothelium regulates vascular homeostasis by elaborating a variety of paracrine factors that act locally in the blood vessel wall and lumen. Under normal conditions, these aspects of the endothelium, hereinafter referred to as “endothelial factors”, maintain normal vascular tone, blood fluidity, and limit vascular inflammation and smooth muscle cell proliferation. Endothelial dysfunction causes impaired vascular reactivity, compounds the adverse effects of inflammatory factors, and underlies a variety of vascular and non-vascular diseases, particularly heart attack and stroke.

Prior art means for estimating endothelial dysfunction include the use of cold pressure tests by invasive quantitative coronary angiography and the injection of radioactive material and subsequent tracking of radiotracers in the blood. These invasive methods are costly, inconvenient, and must be administered by highly trained medical practitioners. Noninvasive prior art methods for measuring endothelial dysfunction include, the measurement of the percent change and the diameter of the left main trunk induced by cold pressure test with two dimensional echo cardiography, the Dundee step test, laser doppler perfusion imaging and iontophoresis, and high resolution lo-mode ultrasound.

Brachial artery imaging with high-resolution ultrasound during an arm-cuff occlusion reactive hyperemia test (flow-mediated vasodilatation, FMD) is now a widely used method of determining peripheral vascular function. Arm cuff inflation provides a suprasystolic pressure stimulus. Ischemia reduces distal resistance and opening the cuff induces stretch in the artery. Imaging of the diameter of the artery along with measuring the peak flow defines endothelial function. The problems and difficulties associated with the ultrasound imaging such as sensitivity to probe positioning, signal artifacts, poor repeatability, need for skilled technicians, observer dependence, observation bias, and high cost have limited the use of this invaluable test to research laboratories.

The present inventors have developed and described Digital Thermal Monitoring (DTM) as a new surrogate for endothelial function monitoring. (See PCT/US2005/018437, published as WO05/118516, incorporated herein by reference). DTM entails measuring temperature changes at the fingertips during arm-cuff occlusion and subsequent reactive hyperemia. However, in some individuals increased sympathetic nervous system activity can interfere with digital thermal monitoring. What is needed are methods and apparatus for identifying aberrant responses due to increased sympathetic nervous system activity and evaluating endothelial function in individuals exhibiting this response.

### SUMMARY OF THE INVENTION

The disclosures herein relate generally to vascular health and neurovascular conditions and more particularly to a method and apparatus for determining one or more health conditions.

It is emphasized that this summary is not to be interpreted as limiting the scope of these inventions which are limited only by the claims herein.

### BRIEF DESCRIPTION OF THE DRAWINGS

FIG. 1 is a schematic view illustrating an exemplary embodiment of a system for determining one or more health conditions, including computer system, database, thermal energy sensor and vasostimulant in relation to a subject.

FIG. 2 is a perspective view illustrating an exemplary embodiment of an apparatus implementing the system of FIG. 1.

FIG. 3 is a flow chart illustrating an exemplary embodiment of the function of a thermal energy sensor engine and vasostimulant engine used in the system of FIG. 1.

FIG. 5 is a flow chart illustrating an exemplary embodiment of the function of a plotting engine used in the system of FIG. 1.

FIG. 6a is a perspective view illustrating an exemplary embodiment of an apparatus for determining one or more health conditions. FIG. 6b illustrates a toe implemented version of one embodiment of the invention. FIG. 6c illustrates an embodiment of a temperature sensor for a digit.

FIG. 7 is a perspective view illustrating an exemplary embodiment of the subject of FIG. 1 coupled to the apparatus of FIGS. 6a.

FIG. 8 is a perspective view illustrating an exemplary embodiment wherein thermal energy sensors are disposed in a glove including sensors on the palm and radial artery.

FIG. 9 is a representative graph of temperature changes at the fingertip during brachial artery hyperemia induced by cuff inflation (two minutes) and deflation.

FIG. 10 is a graph illustrating an exemplary experimental embodiment of temperature vs. time data obtained including DTM of a test finger and a contralateral corresponding control finger using the apparatus of FIGS. 1 and 2 and using the method of FIG. 4-5.

FIG. 11 is a graph illustrating an exemplary experimental embodiment of temperature vs. time data obtained including DTM of a test finger and a contralateral corresponding control finger of the same individual as in FIG. 10 but where the starting finger tip temperature is less than 26° C.

FIG. 12 is a graph illustrating an exemplary experimental embodiment of temperature vs. time data obtained including DTM of a test finger and a contralateral corresponding control finger of an individual exhibiting a steady declining temperature of the control finger.

FIG. 13 is a graph illustrating an exemplary experimental embodiment of temperature vs. time data obtained including DTM of a test finger and a contralateral corresponding control finger of an individual exhibiting a steady increasing temperature of the control finger.

FIG. 14 is a perspective view illustrating an exemplary embodiment including a Doppler probe.

FIG. 15 is a perspective view illustrating the exemplary embodiment including a Doppler probe of FIG. 14 placed on a patient.

FIG. 16 is a graph illustrating an experimental embodiment of the apparatus of FIG. 14 and 15 showing the Doppler data.

FIG. 17 depicts an embodiment for digital and/or palm temperature monitoring including Doppler detection at the radial artery.

FIG. 18 depicts results from thermal imaging before (A), during (B) and after (C) cuff occlusion.

FIG. 19 depicts a combination of DTM (A), Doppler (B) and thermal imaging (C) of the forearm.

### DETAILED DESCRIPTION

A method for isolating endothelial function from neurovascular is provided. The present inventors have determined that skin temperature in a digit distal (Digital Temperature Monitoring) to the site of occlusion can be used to evaluate microvascular endothelial function in the context of reactive hyperemia. Reactive hyperemia represents transient endothelial-mediated vasodilation following restoration of blood flow after occlusion in persons with normal endothelial function. However, vasodilation has both local endothelial and neurovascular components. DTM reflects microvascular reactivity at skin level and also to some degree neurovascular response or reactivity mediated by the autonomic nervous system (ANS). In measurement of vascular reactivity, researchers usually attempt to discount the neurovascular component and describe it as noise. In order for Digital Temperature Monitoring (DTM) to accurately reflect the response of the endothelium to hyperemia, neurovascular contributions should ideally be identified and controlled if possible in the individual patient.

After occlusion of blood flow in a limb, the skin temperature of a digit distal to the site of occlusion will drop steadily. After blood flow is restored, reactive hyperemia will result the skin temperature rebounding. In persons will good vascular function and reactivity, the skin temperature will rebound to a level higher than an equilibrated temperature prior to occlusion. However, vasospasticity mediated by neurovascular activity can obscure the ability of DTM to accurately measure the response of the vascular reactivity to hyperemia. Even in normal individuals, cold will induce shunting of blood away from the periphery. In certain individuals, neurovascular responses have been observed that are particularly difficult to control. This response has been termed “cold finger.” The present invention provides methods and apparatus for identifying and minimizing neurovascular interferences such that an accurate status of vascular reactivity can be obtained.

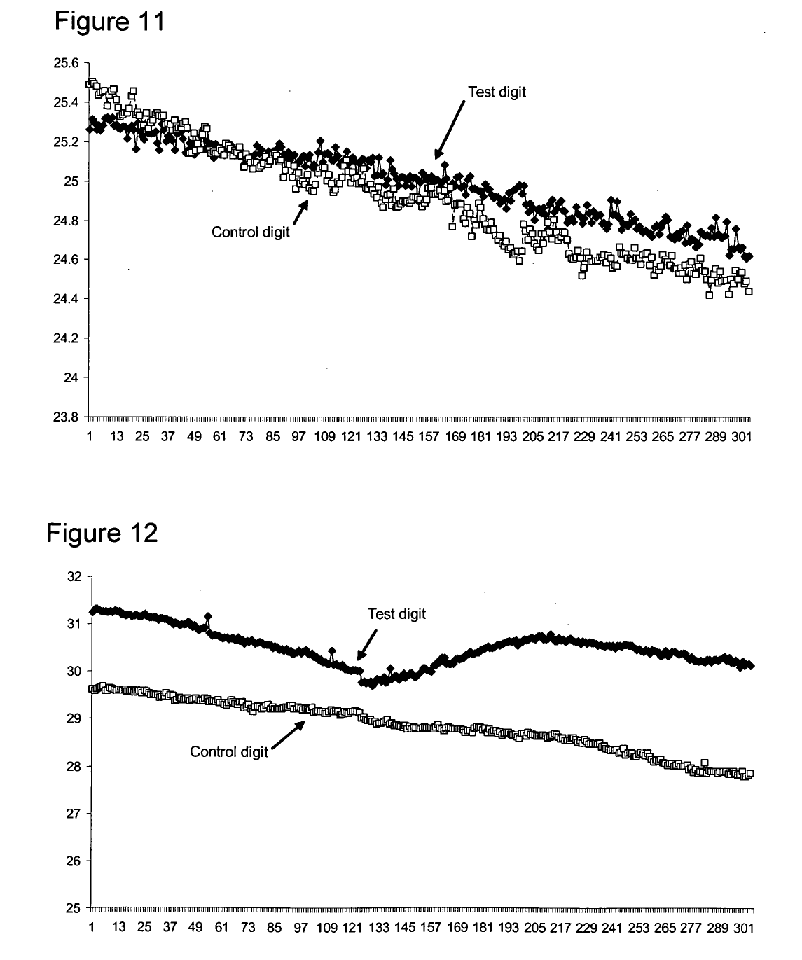

Cold finger is a manifestation of excessive sympathetic nervous system activity. This neurovascular response, if systemic or symmetric, is expected to reflect both limbs (the occluded and the contralateral limb). An ideal scenario would for DTM would be to have minimum changes in the contralateral finger. FIG. 10 reflects such a situation where the temperature in the control corresponding contralateral digit is essentially stable throughout the procedure. Cold finger interferes with this goal. As depicted in FIG. 11, the same individual as depicted in FIG. 10 exhibited a vasospastic response of sufficient magnitude when her hands were cold that no temperature rebound could be detected. To avoid such phenomena, the DTM procedure is preferably operated within a preset fingertip temperature range to control for vasospasticity.

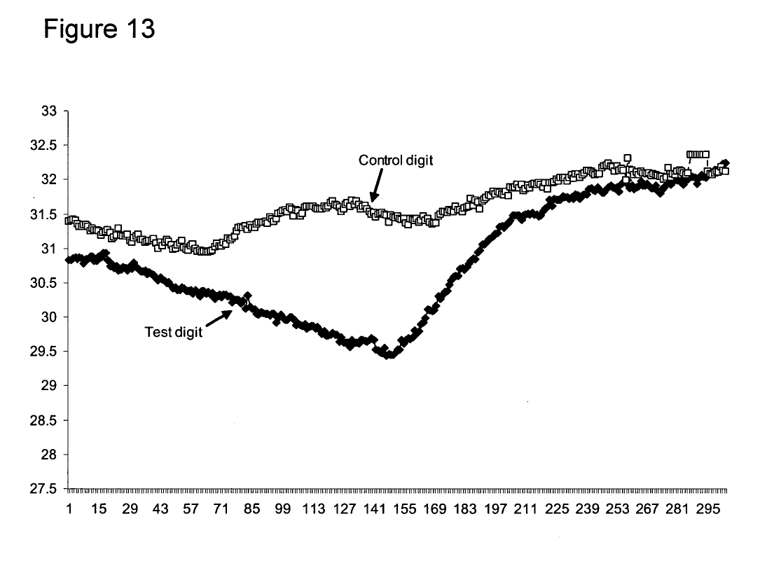

In one embodiment, a start temperature lower than 28° C. is considered less desirable and the patient is asked to wait and relax to warm up in order to reduce the effect of ANS and sympathetic overshoot in measuring vascular reactivity by DTM and/or Doppler flow. This period allows for control of high basal ANS (sympathetic) activity that is associated with mental stress and anxiety such as white coat hypertension. Other individuals respond to the DTM monitoring with an increased (FIG. 13) or decreased temperature (FIG. 12) in the contralateral finger. Decline in the fingertip temperature of occluded arm after releasing the cuff is also considered to be due to excessive sympathetic activity and such results are treated as a bad test that must be repeated. Such responses are identified by a computer implemented program that is designed to flag a negative NP.

In one embodiments, the palm is used as a site of temperature monitoring as the palm is considered relatively less susceptible to neurovascular influence. A differential finger-palm temperature response may be considered an indicator of ANS activity.

DTM monitoring can measure microvascular reactivity controlling the amount of blood flow in a given tissue. However, DTM measures a delayed signal from microvascular reactivity the skin level and includes a neurovascular component (sympathetic or anatomic nervous system). In contrast, Doppler measurement of flow, for example through the radial artery, provides a measure of flow through the entire distal microvasculature and combines deep tissue microvascular reactivity and superficial. The Doppler signal is rapid and is less affected by neurovascular response.

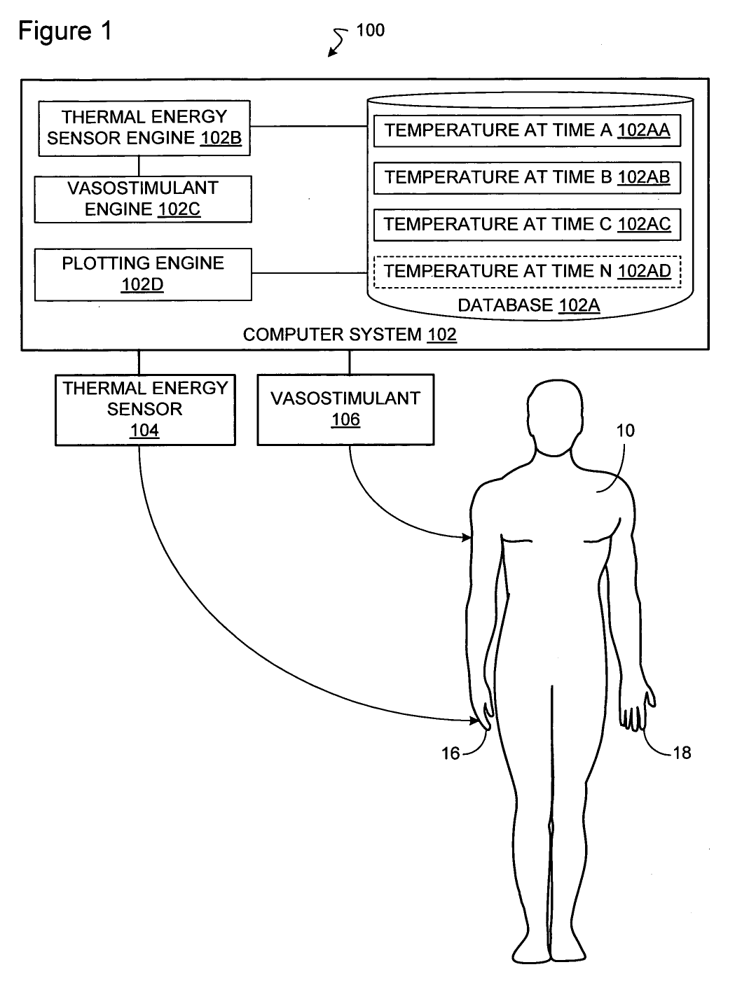

Referring to FIG. 1, in one embodiment, an apparatus for determining one or more health conditions 100 includes a computer system 102 which is operably coupled to a thermal energy sensor 104 and a vasostimulant 106. The computer system 102 may be, for example, a conventional computer system known in the art. The thermal energy sensor 104 may be a conventional thermal energy sensor known in the art. In an exemplary embodiment, the thermal energy sensor 104 may be, for example, a thermocouple, a thermister, a resistance temperature detector, a heat flux sensor, a liquid crystal sensor, an infrared sensor, a thermopile, or a variety of other thermal energy sensors known in the art. In one embodiment, the thermal energy sensor is an infrared sensor that measures the thermal energy of a point or of an area on a surface. In one embodiment, the thermal energy sensor 104 may be disposable.

The vasostimulant 106 may be, for example, conventional vasostimulants known in the art including mechanical vasostimulants such as cuffs for compressing arteries. In one embodiment, the thermal energy sensor 104 and the vasostimulant 106 are coupled to, monitored by, and/or controlled by the computer system 102 through a wireless connection such as, for example, a wireless connection including BLUETOOTH wireless technology. In an exemplary embodiment, the computer system 102 may be coupled to a variety of convention medical devices known in the art such as, for example, a conventional pulse oximeter or a conventional blood pressure monitoring device.

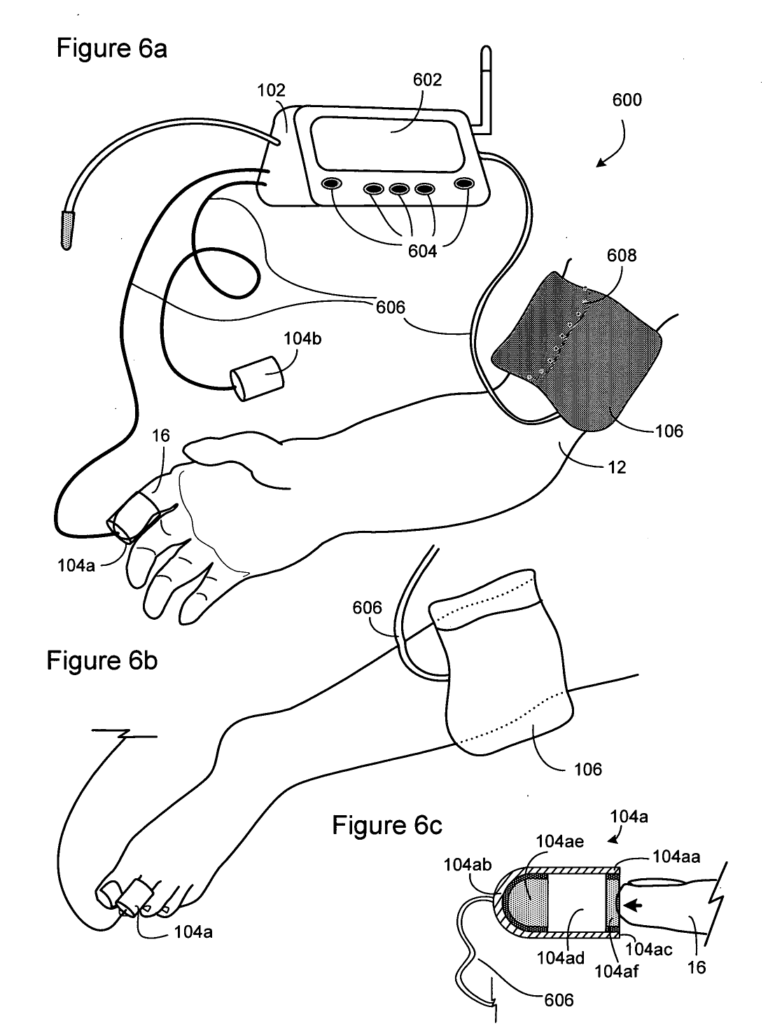

In an exemplary embodiment, the computer system includes a database. A thermal energy sensor engine is operably coupled to the database. A vasostimulant engine is operably coupled to the database and the thermal energy sensor engine. A plotting engine is operably coupled to the database. In an exemplary embodiment, the thermal energy sensor engine, vasostimulant engine, and the plotting engine may be, for example, a variety of conventional software engines known in the art. In several exemplary embodiments, the thermal energy sensor engine is adapted to control a thermal energy sensor, which is operably coupled to the computer system. In several exemplary embodiments, the vasostimulant engine 102C is adapted to control a vasostimulant such as, for example, the vasostimulant 106 illustrated in FIGS. 6a, which is operably coupled to computer system 102. Referring to FIG. 1, in one embodiment, the database 102A includes a plurality of data such as, for example, a temperature at time A 102AA, a temperature at time B 102AB, a temperature at time C 102AC, up to a temperature at time N 102AD. In an exemplary embodiment, the temperature data may include temperatures taken from one thermal energy sensor such as, for example, the thermal energy sensor 104a illustrated in FIGS. 6a and b, or from a plurality of thermal energy sensors.

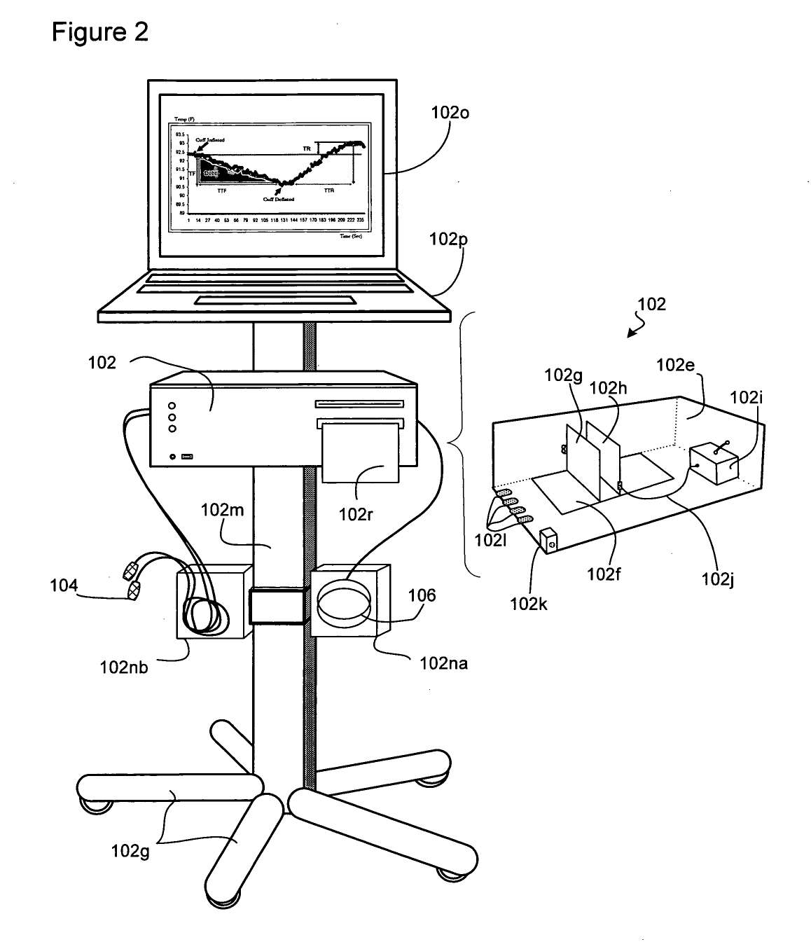

Referring now to FIG. 2, in an exemplary embodiment, the computer system 102 includes a chassis 102e. A computer board 102f is mounted to the chassis 102e and includes a thermal energy sensor card 102g and a vasosimulant card 102h coupled to and extending from the computer board 102f. A pump 102i is coupled to the vasostimulant card 102h by a wire 102j. The pump may or may not be internal to the computer chassis 102e. In an exemplary embodiment, the chassis 102e may include wireless interface 102k for allowing wireless communication to the computer board 102f. In an exemplary embodiment, the chassis may include a plurality of communications ports 102l mounted to a surface for allowing communication with the computer board 102f. In an exemplary embodiment, the thermal energy sensor card 102g is coupled to the thermal energy sensor 104a, illustrated in FIGS. 6a and b. In an exemplary embodiment, the vasostimulant card 102h is coupled to the vasostimulant 106, illustrated in FIGS. 6a and b, through the pump 102i.

Referring again to FIG. 2, in an exemplary embodiment, the computer system 102 is positioned on a chassis 102m. A plurality of storage units 102na and 102nb extend from opposite sides of the chassis 102m with the storage unit 102na providing storage for the vasostimulant 106, and the storage unit 102nb providing storage for the thermal energy sensor 104. A display 102o is mounted to and positioned on top of the chassis 102m and coupled to the computer system 102 in order to display data collected by the computer system 102. An input device 102p is mounted to the chassis 102m to provide input the computer system 102 and manipulate information displayed on the display 102o. In an exemplary embodiment, the chassis 102m includes a plurality of wheels 102q which are operable to allow moving of the chassis 102m. In an exemplary embodiment, the computer system 102 is operable to produce an output 102r which includes data collected by the computer system 102.

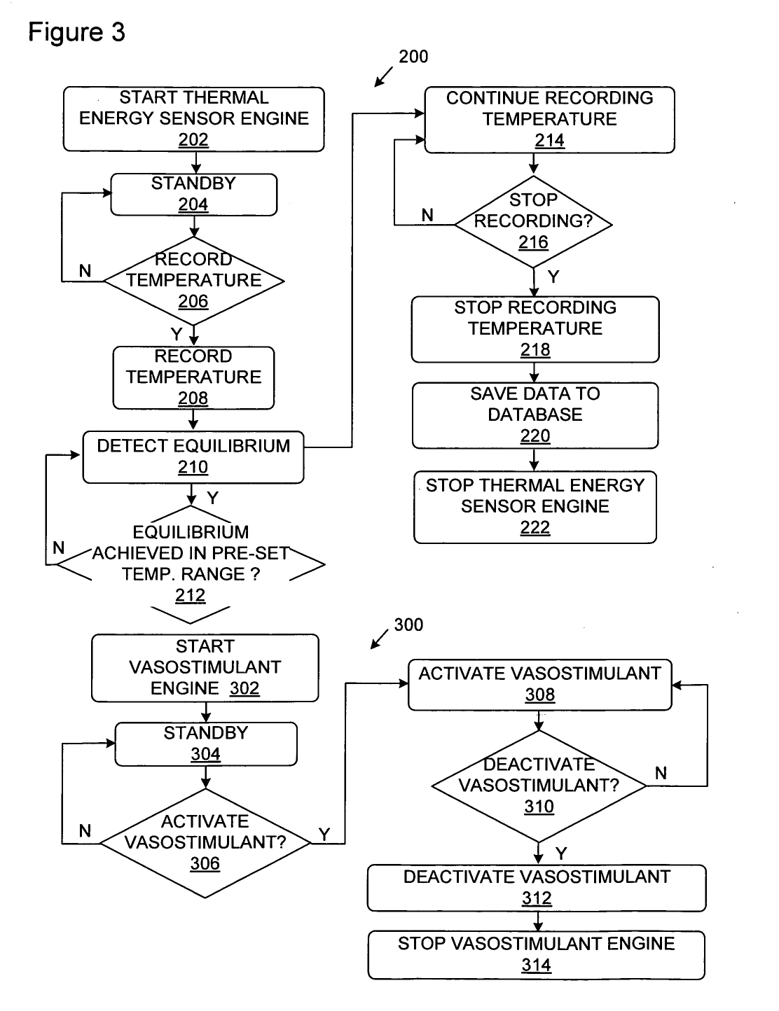

Referring now to FIG. 3, in an exemplary embodiment, a method 200 for controlling a thermal energy sensor is illustrated in which a thermal energy sensor engine such as, for example, the thermal energy sensor engine 102b illustrated in FIG. 1, is started in step 202. Starting the thermal energy sensor engine 102b at step 202 allows the thermal energy sensor engine 102b to enter a standby mode at step 204. At decision block 206, the thermal energy sensor engine 102b determines whether it is time to start recording temperature with a thermal energy sensor. If it is not time to start recording temperature, the method 200 returns to step 204 where the thermal energy sensor engine 102b remains on standby.

If it is time to start recording temperature, the thermal energy sensor engine 102b begins recording temperature at step 206 with the thermal energy sensor 104. The method 200 then proceeds to step 208 where the thermal energy sensor engine 102b begins to detect for temperature equilibrium in step 210. In an exemplary embodiment, at step 210, the thermal energy sensor engine begins comparing successive temperature measurements made by the thermal energy sensor 104. At decision block 212, the thermal energy sensor engine 102b determines whether temperature equilibrium has been achieved in a preset temperature range. In one embodiment, the present temperature range is at a middle area between room temperature, typically 25-26° C. and core body temperature, typically 35-36° C. Thus, in one embodiment the optimum fingertip temperature is approximately 31-32° C. The present range will not permit the process to proceed if the fingertip temperature is outside of the present range. In one embodiment, the minimum for the present range is approximately 27° C. In another embodiment, the minimum for the present range is approximately 28° C. to avoid neurovascular influences. In an exemplary embodiment, temperature equilibrium is achieved when temperature changes recorded by the thermal energy sensor 104 are less than 0.1 degrees C. If the equilibrium has not been achieved, the method 200 returns to step 210 where the thermal energy sensor engine 102b detects for temperature equilibrium.

If equilibrium has been achieved, the method 200 proceeds to step 214 where the thermal energy sensor engine 102b continues recording temperature measurements made by the thermal energy sensor 104. At decision block 216, the thermal energy sensor engine 102b determines whether to stop recording. In an exemplary embodiment, the thermal energy sensor engine 102b will stop recording when temperature measurements from the thermal energy sensor 104 have stabilized. If it is not time to stop recording, the method 200 returns to step 214 where the thermal energy sensor engine 102b continues recording temperature measurements made by the thermal energy sensor 104.

If it is time to stop recording, the method 200 proceeds to step 218 where the thermal energy sensor engine 102b stops recording temperature measurements made by the thermal energy sensor 104. The method then proceeds to step 220 where the temperature measurements recorded by the thermal energy sensor engine 102b are saved to a database such as, for example, the database 102a illustrated in FIG. 1. The method 200 then proceeds to step 222 where the thermal energy sensor engine 200 is stopped.

Referring again to FIG. 3, in an exemplary embodiment, vasostimulant engine 300 such as, for example, the vasostimulant engine 102c illustrated in FIG. 1, is started in step 302. Starting the vasostimulant engine 102c at step 302 allows the vasostimulant engine 102c to enter a standby mode at step 304. At decision block 306, the vasostimulant engine 102c determines whether to activate a vasostimulant such as, for example, the vasostimulant 106 illustrated in FIGS. 6a. If it is not time to activate the vasostimulant 106, the method 300 returns to step 304 where the vasostimulant engine 300 remains on standby.

If it is time to activate the vasostimulant 106, the method 300 proceeds to step 308 where the vasostimulant engine 102c activates the vasostimulant 106. At decision block 310, the vasostimulant engine 102c determines whether it is time to deactivate the vasostimulant 106. If it is not time to deactivate the vasostimulant 106, the method 300 returns to step 308 where the vasostimulant engine 102c keeps the vasostimulant 106 activated.

If it is time to deactivate the vasostimulant 106, the method 300 proceeds to step 312 where the vasostimulant engine 102c deactivates the vasostimulant 106. The method 300 then proceeds to step 314 where the vasostimulant engine 102c is stopped.

Referring now to FIG. 4, in one embodiment, a method for controlling a plotting engine 400 is illustrated in which a plotting engine such as, for example, the plotting engine 102d illustrated in FIG. 2, is started in step 402. Starting the plotting engine 102d at step 402 allows the plotting engine 102d to enter a standby mode at step 404. At decision block 406, the plotting engine 102d determines whether it is time to plot data. If it is not time to plot data, the method 400 returns to step 404 where the plotting engine 102d remains on standby.

If it is time to plot data, the method 400 proceeds to step 408 where the plotting engine 102d retrieves data from a database such as, for example, the database 102a illustrated in FIG. 1. At decision block 410, the plotting engine 102d determines whether all of the data needed has been retrieved from database 102a. If all the data has not been retrieved, the method 400 returns to step 408 where the plotting engine 102d continues to retrieve data from database 102a.

If all the data needed has been retrieved from database 102a, the method proceeds to step 412 where the plotting engine 102d plots the data. The method 400 then proceeds to step 414 where the plotting engine 102d is stopped.

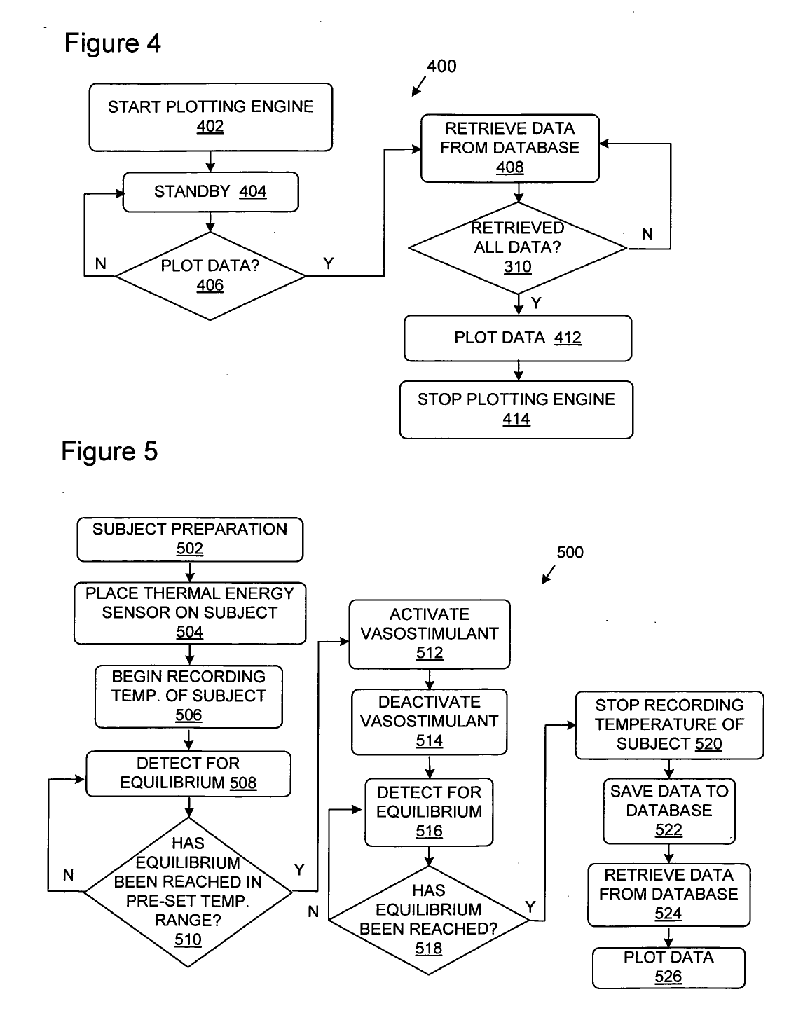

Referring to FIG. 5, in an exemplary embodiment, a method for determining one or more health conditions 500 is illustrated which begins with a subject preparation at step 502. Subject preparation at step 502 may include, for example, having a subject such as, for example, the subject 10 illustrated in FIG. 1, refrain from certain activities before carrying out the method 500, such as eating, smoking, ingesting alcohol or caffeine, taking any vascular medications, experiencing urinary urgency or full bladder, exposure to cold weather, physical or mental exercise, and a variety of other factors that may temporarily affect vascular function known in the art. In one embodiment, the subject preparation at step 502 may begin at least 12 hours prior to the method 500 proceeding to step 504.

At step 504, a thermal energy sensor such as, for example, the thermal energy sensor 104 illustrated in FIGS. 6a-c, may be placed on the subject 10. In an exemplary embodiment, the thermal energy sensor 104 may be a conventional thermal energy sensor known in the art. In an exemplary embodiment, the thermal energy sensor 104 is designed such that there is a minimal area of contact between the sensor and the subject 10. In an exemplary embodiment, when placed on the subject 10, the thermal energy sensor 104 provides a minimal pressure to the subject 10, measures thermal energy only and does not introduce any signals into the subject 10 or alter the microcapillary flow. In an exemplary embodiment, a plurality of thermal energy sensors 104 may be positioned at different locations on the subject 10. In an exemplary embodiment, one thermal energy sensor 104 is positioned on a test digit and a second thermal energy sensor is placed on a contralateral control digit on a contralateral limb.

In an exemplary embodiment, the thermal energy sensor may be placed on the subject in order to measure the thermal energy of distal resistant vessels on the subject. In an exemplary embodiment, the thermal energy sensor 104 may allow the visualization of thermal response by infrared thermal energy measuring devices such as, for example, cameras, thermosensors, and/or thermocouples. In an exemplary embodiment, the thermal energy sensor 104 minimizes the temperature changes associated with the contact of the skin surface and thermal energy sensor 104 and allows the thermal energy sensor 104 to be minimally effected by factors and conditions that change skin temperature but are not associated with changes in blood flow, subcutaneous blood flow, tissue heat generation, and/or tissue heat transduction.

At step 506, a thermal energy sensor engine such as, for example, the thermal energy sensor engine 102b illustrated in FIG. 1, activates a thermal energy sensor such as, for example, the thermal energy sensor 104 illustrated in FIG. 6c, to begin recording the temperature of the subject 10. In an exemplary embodiment, temperature data begins being recorded continuously. In an exemplary embodiment, the thermal energy sensor 102b measures the skin temperature of the subject's body on which it is placed such as, for example, a digit of a hand or foot. In an exemplary embodiment, the ambient temperature is held constant around the thermal energy sensor 104.

At step 508, the thermal energy sensor engine 102b begins to detect for equilibrium in the temperature of subject 10. In an exemplary embodiment, at step 508, the thermal energy sensor engine 102b retrieves successive temperature measurements from the thermal energy sensor 104.

At decision block 510, the thermal energy sensor engine 102b determines whether the temperature of the subject 10 has reached equilibrium. At decision block 510, the thermal energy sensor engine 102b determines whether temperature equilibrium has been achieved in a preset temperature range. In one embodiment, the present temperature range is at a middle area between room temperature, typically 25-26° C. and core body temperature, typically 35-36° C. Thus, in one embodiment the optimum fingertip temperature is approximately 31-32° C. The present range will not permit the process to proceed if the fingertip temperature is outside of the present range. In one embodiment, the minimum for the present range is approximately 27° C. In another embodiment, the minimum for the present range is approximately 28° C. to avoid neurovascular influences. If the temperature of the subject 10 has not reached equilibrium or is outside of the reset range, the temperature sensor engine proceeds back to step 508 to detect for equilibrium. In an exemplary embodiment, determining whether the temperature of the subject 10 has reached equilibrium in step 510 may include, for example, determining whether the temperature changes of a subject 10 are less than 0.1 degree C.

If the temperature changes in the subject 10 have reached equilibrium, the method proceeds to step 512 where a vasostimulant engine such as, for example, the vasostimulant engine 102c illustrated in FIG. 1, activates a vasostimulant such as, for example, the vasostimulant 106 illustrated in FIG. 6a or 6b. In an exemplary embodiment, the vasostimulant 106 may be an inflatable cuff, and activating the vasostimulant 106 at step 512 may include, for example inflating the cuff to 200 mm Hg systolic BP. The continued recording of temperature may then include visualizing the thermal response of the subject 10 with an infrared thermal measurement device.

At step 514, the vasostimulant engine 102c may deactivate the vasostimulant 106 and where the vasostimulant 106 is an inflatable cuff, deactivating the vasostimulant 106 at step 514 deflates the cuff. In an exemplary embodiment, the vasostimulant is deactivated anywhere from 2 to 5 minutes after activation in step 512. In an exemplary embodiment, the vasostimulant is deactivated at less than 5, 4, 3 or 2 minutes after activation in step 512, which is less than the conventional deactivation time for tests involving vasostimulation and provides a method which reduces the pain sometimes associated with vasostimulants. At step 516, the thermal energy sensor engine 102b begins to detect for equilibrium in the temperature of subject 10. In an exemplary embodiment, at step 516, the thermal energy sensor engine 102b retrieves successive temperature measurement from the thermal energy sensor.

At decision block 518, the thermal energy sensor engine 102b determines whether the temperature of the subject 10 has reached equilibrium. If the temperature of the subject 10 has not reached equilibrium, the temperature sensor engine proceeds back to step 516 to detect for equilibrium. In an exemplary embodiment, determining whether the temperature of the subject 10 has reached equilibrium in step 518 may include, for example, determining whether the temperature changes of a subject 10 are less than 0.1 degree C.

If the temperature changes in the subject 10 have reached equilibrium, the method proceeds to step 520 where the temperature sensor engine 102b stops recording the temperature of the subject 10.

At step 522, data acquired from measuring and recording temperature changes which began at step 506 and continued throughout the method 500 is saved by the temperature sensor engine 102b to a database such as, for example, the database 102a illustrated in FIG. 1.

At step 524, a plotting engine such as, for example, the plotting engine 102d illustrated in FIG. 1, may retrieve data from the database 102a.

At step 526, the plotting engine 102d may plot out the data retrieved. In an exemplary embodiment, the data may be plotted out as temperature vs. time. In an exemplary embodiment, the plotting engine 102d may plot out data obtained from the temperature measurements concurrent with the data being obtained. In an exemplary embodiment, the plotting engine 102d may retrieve data taken from multiple positions on subject 10 and plot out an average of that data over time. In an exemplary embodiment, the plotting engine 102d may retrieve data taken from subject 10 at different times and plot out an average of that data.

Referring now to FIG. 6a, an alternative embodiment of an apparatus for determining one or more health conditions 600 is substantially identical in design and operation to apparatus 100 described above with reference to FIGS. 1-2 with the addition of a display 602, a plurality of output buttons 604, a plurality of coupling wires 606, and vasostimulant coupling member 608. Computer system 102 includes the display 602 and the plurality of display output buttons 604 on a surface. A plurality of the thermal energy sensors 104a and 104b are coupled to the computer system 102 by respective coupling wires 606. The vasostimulator 106 is a pressure cuff and is coupled to the computer system 102 by coupling wire 606. The pressure cuff vasostimulator 106 includes a vasostimulant coupling member 608 along an edge of its length. In an exemplary embodiment, the pressure cuff vasostimulator 106 may be adapted to measure a subject's blood pressure.

Thermal energy sensor 104a is substantially similar to thermal energy sensor 104b and, referring to FIG. 6c, in one embodiment includes a tubular housing 104aa with a hemispherical closed end 104ab and an open end 104ac opposite the closed end 104ab. The housing 104aa defines a passageway 104ad therein, and includes a thermal energy measurement device 104ae positioned in the passageway 104ad and adjacent the closed end 104ab. A coupling member 104af is positioned in the passageway 104ad adjacent the open end 104ac.

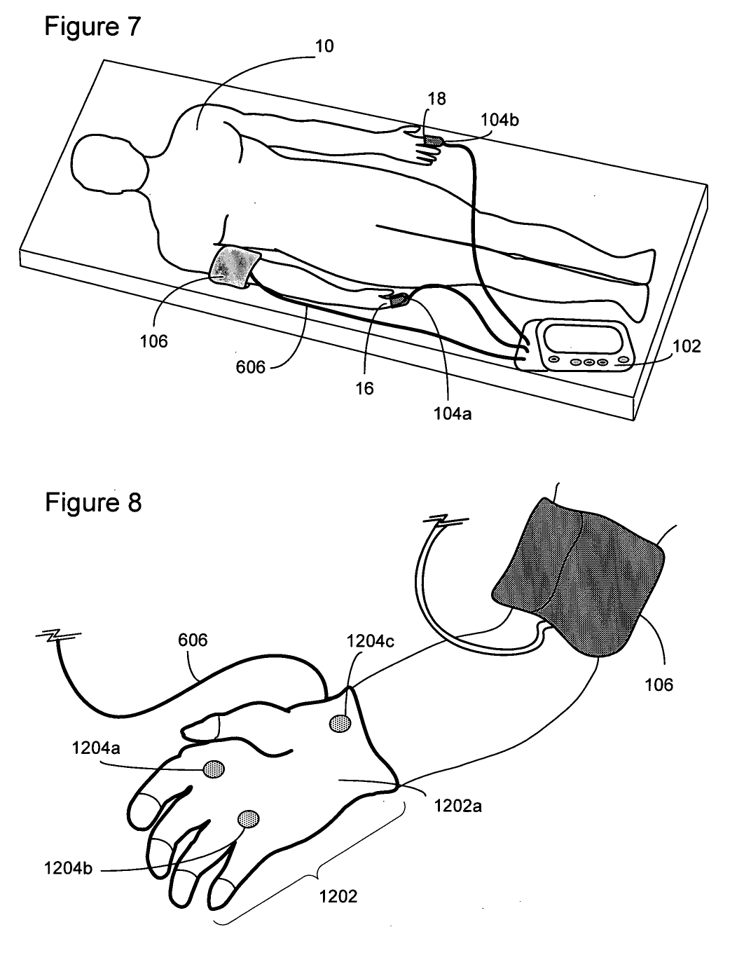

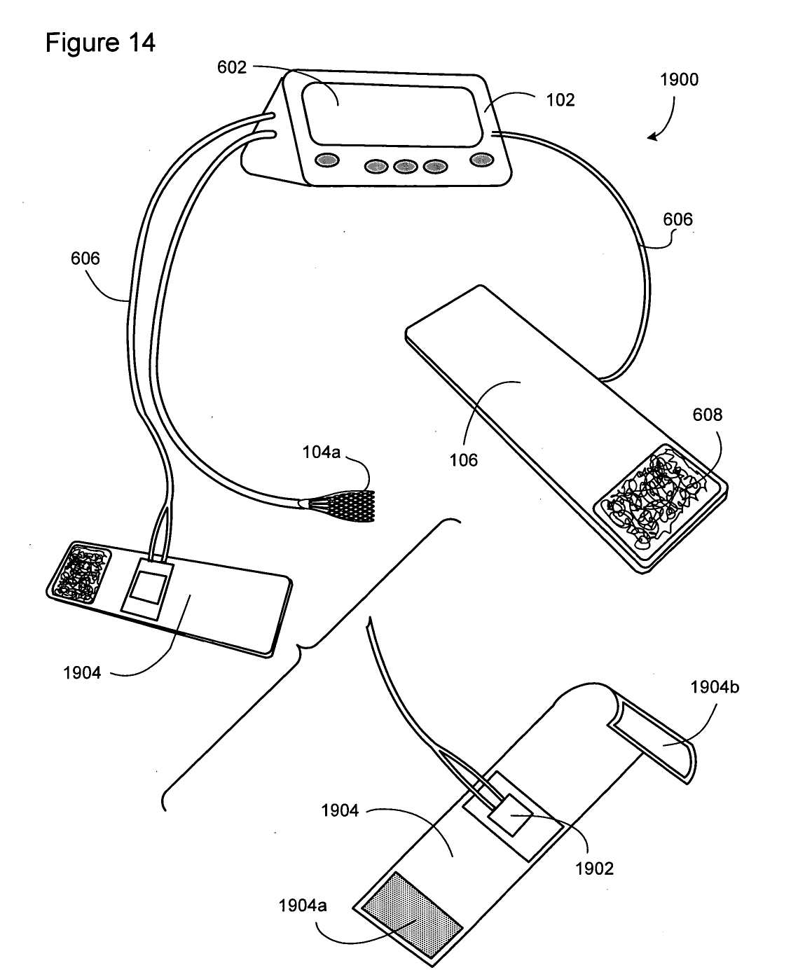

Referring to FIG. 7, a method for determining one or more health conditions is illustrated in which subject preparation begins with placing the pressure cuff vasostimulant 106 on a limb of subject 10. Pressure cuff vasostimulant 106 may be secured to arm 12 by vasostimulant coupling member 608 as depicted in FIG. 14 which may include a variety of adhesive materials known in the art. In an exemplary embodiment, the subject may be in a prone or seated position during the procedure.

In one embodiment, a further step is included after step 504 of FIG. 6, in which a further thermal energy sensor 104b is placed on contralateral corresponding digit 18 of the subject 10. The contralateral digit 18 is placed in thermal energy sensor 104b in substantially the same manner as finger 16 is placed in thermal energy sensor 104a. In an exemplary embodiment, a plurality of thermal energy sensors, may be placed on a plurality of contralateral body parts. In an exemplary embodiment, a contralateral body part includes any body part on the subject 10 which is not directly affected by the vasostimulant activated in step 512 such as, for example, any body part on the subject 10 which is not distal to the vasostimulant. In an exemplary embodiment, and as depicted in FIG. 7a, the thermal energy sensor 104a is placed on a finger 16 which is distal to and directly affected by the action of the vasostimulant, while thermal energy sensor 104b is placed on contralateral finger 18 and/on a toe of the subject. As used herein, “corresponding” digit refers to the same finger or toe on the contralateral limb.

At step 506, a thermal energy sensor engine such as, for example, the thermal energy sensor engine 102b illustrated in FIG. 1, activates the thermal energy sensors 104 to begin recording the skin temperature of the finger 16 and contralateral finger 18 or a toe of subject 10. In an exemplary embodiment, temperature data begins being recorded continuously. The method proceeds essentially in accordance with the description of method 500.

In an exemplary embodiment, the data for the finger 16 and contralateral finger 18 are plotted on the same graph as depicted in FIG. 10. In an exemplary embodiment, the plotting engine 102d may plot out data obtained from the temperature measurements concurrent with the data being obtained. In an exemplary embodiment, the temperature changes measured in the finger 16 may be adjusted based on the temperature changes measured in the contralateral finger 18. For example, the adjustment may include subtracting the temperature changes measured in the contralateral finger 18 from the temperature changes measured in the finger 16, or vice versa.

In one embodiment, as depicted in FIG. 6b, a method for determining one or more health conditions begins with placing the pressure cuff vasostimulant 106 on a leg of subject 10 at step 502. Pressure cuff vasostimulant 106 may be secured to the leg by vasostimulant coupling member 608 which may include a variety of adhesive materials known in the art.

At step 504, thermal energy sensor 104a may be placed on a toe of the subject 10. A toe is placed in thermal energy sensor 104b in substantially the same manner as finger 16 is placed in thermal energy sensor 104a described above with reference to FIG. 7. The method proceeds essentially in accordance with the description of method 500.

Referring now to FIG. 8, an alternative embodiment of an apparatus for determining one or more health conditions 500 is substantially identical in design and operation to apparatus 600 described above with reference to FIG. 6a, with the addition of a thermal energy sensor 1202. Thermal energy sensor 1202 is coupled to computer system 102 by wire 606 and includes a glove 1202a including a plurality of thermal energy measurement devices 1204a, 1204b, and 1204c, which are positioned at different locations on the glove 1202a. Having the thermal energy measurement devices 1204a, 1204b, and 1204c positioned at different locations on the glove 1202a allows blood flow rate from device to device to be calculated. In an exemplary embodiment, glove 1202a may extend and cover the skin surface up to the vasostimulant 106.

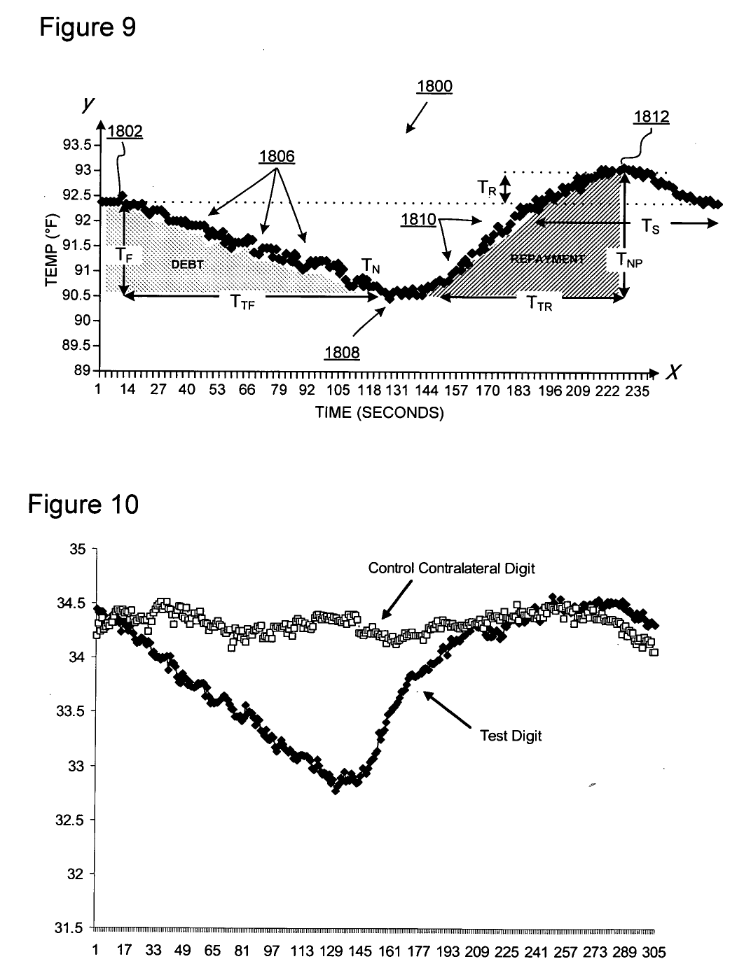

Referring now to FIG. 9, a representative experimental graph of temperature vs. time is illustrated for a healthy subject during the method 500. In an exemplary embodiment, the graph may be produced by the plotting engine 102d, illustrated in FIG. 1. A baseline temperature 1802 is achieved when the subject reaches a steady temperature after having a thermal energy sensor such as, for example, the thermal energy sensor 104 illustrated in FIG. 2 and 6a-c, coupled to them. At time 1804, the vasostimulant is activated, causing the temperature of the subject to drop, resulting in a debt slope (SD) 1806. At time 1808, the vasostimulant is deactivated, causing the temperature of the subject to rise, resulting in a repayment slope (SR) 1810. The temperature of the subject crosses the baseline temperature 1802 and reaches a peak temperature 1812, after which the temperature returns back to the baseline temperature 1802. A number of measurements can be made from the data shown in graph including, but not limited to, the fall temperature change T_{F} between the baseline temperature 1802 and the temperature recorded at time 1808, the rebound temperature change T_{R} between the baseline temperature 1802 and the peak temperature 1812, the nadir to peak temperature change T_{NP} between the temperature recorded at time 1808 and the peak temperature 1812, the time to fall temperature T_{TF}, the time to rebound temperature T_{TR}, the time to stabilized temperature T_{S}, the steepness of the slopes (S_{D}) 1806 and (S_{R}) 1810, the area under the temperature curve bounded by the temperature curve and the temperature reached at time 1808 and between time equal zero and time 1808, the area under the temperature curve bounded by the temperature curve and the temperature reached at time 1808 and between time 1808 and the time at peak temperature 1812, and the area under the temperature curve bounded by the temperature curve and the temperature reached at time 1808 and between time 1808 and the time at which the temperature stabilizes.

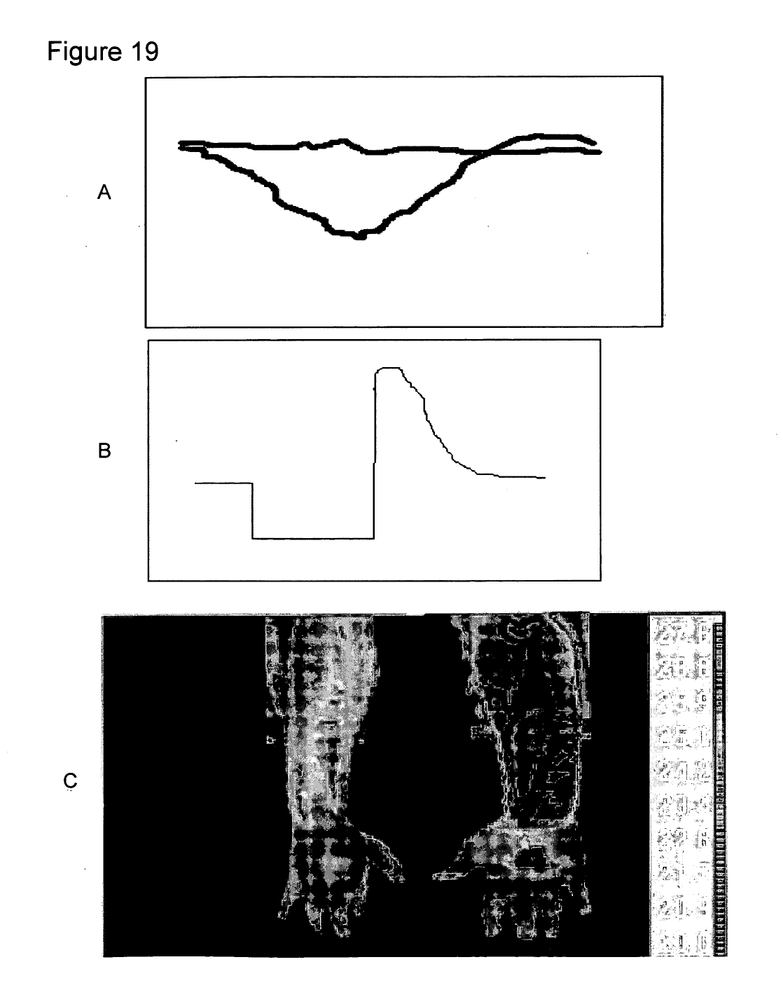

In an exemplary embodiment, healthy vascular reactivity as depicted in FIG. 19 may be indicated by a value of T_{NP} which is greater than T_{F}. In an exemplary embodiment, unhealthy vascular reactivity may be indicated by a value of T_{NP} which is less than T_{F}. In an exemplary embodiment, unhealthy vascular reactivity may be indicated by a negative value of T_{R}. In an exemplary embodiment, several graphs similar to graph 1800 may be taken from a subject and then averaged to get an average graph for the subject, which may indicate the average response for the subject over a period of time.

In an exemplary embodiment, the value of T_{R} may be normalized using thermodynamic equations for calculating heat flow based on the following parameters: baseline temperature 1802, fall temperature change T_{F}, ambient room temperature, core temperature, tissue heat capacity, tissue metabolism rate, tissue heat conduction, the mass of the testing volume, the location the method is conducted, blood flow rate, the position of the subject 10 during the method, and a variety of other physical and/or physiological factors that may effect the value of T_{R}.

In an exemplary embodiment, determining the status of diabetic foot includes measuring the autonomic nervous systemic function in the subject such as, for example, by looking at the changes in temperature in the contralateral finger 18 on subject 10 after provision of the vasostimulant. In an exemplary embodiment, an increase in temperature in the contralateral finger 18 of subject 10 indicates a healthy autonomic nervous system function in the subject.

In several exemplary embodiments, after acquiring and/or plotting the temperature data obtained using the methods and/or the apparatus of the present invention, additional diagnosis techniques such as, for example, change in Doppler flow in the body part in which temperature is being measured, change in pressure in the body part in which temperature is being measured, and/or change in blood flow measured by near infrared spectroscopy in the body part in which temperature is being measured, may be used to provide a comprehensive determination of health condition of the subject.

In an exemplary embodiment, the determining one or more health conditions for the subject based upon at least one of the temperature changes measured comprises analyzing the temperature response to the vasostimulant in order to identify whether the subject has high sympathetic reactivity. In an exemplary embodiment, the determining one or more health conditions for the subject based upon at least one of the temperature changes measured comprises analyzing the temperature response to the vasostimulant along with additional diagnosis techniques in order to identify whether the subject has high sympathetic reactivity.

In an exemplary embodiment, the determining one or more health conditions for the subject based upon at least one of the temperature changes measured comprises analyzing the temperature response to the vasostimulant in order to screen the subject for white coat hypertension. In an exemplary embodiment, the determining one or more health conditions for the subject based upon at least one of the temperature changes measured comprises analyzing the temperature response to the vasostimulant along with additional diagnosis techniques in order to screen the subject for white coat hypertension.

In an exemplary embodiment, the method further comprises measuring and recording a room temperature. In an exemplary embodiment, the method further comprises measuring and recording a core temperature of the subject. In an exemplary embodiment, the method further comprises measuring and recording a tissue heat capacity of the subject. In an exemplary embodiment, the method further comprises measuring and recording a tissue metabolic rate of the subject.

In an exemplary embodiment, the method further comprises determining a vasodilative index for the subject. In an exemplary embodiment, the method further comprises determining a vasoconstrictive index for the subject. In an exemplary embodiment, the blood pressure of the subject is measured before the provision of the vasostimulant. In an exemplary embodiment, the blood pressure of the subject is measured after the provision of the vasostimulant. In an exemplary embodiment, the blood pressure of the subject is measured before, during, and after the provision of the vasostimulant.

In an exemplary embodiment, the method further comprises measuring the skin temperature changes on a contralateral body part of the subject. In an exemplary embodiment, the contralateral body part comprises a plurality of contralateral body parts. In an exemplary embodiment, the body part is a first hand on the subject, and the contralateral body part is a second hand on the subject. In an exemplary embodiment, the body part is a first foot on the subject, and the contralateral body part is a second foot on the subject. In an exemplary embodiment, the body part is a finger on the subject, and the contralateral body part is a toe on the subject.

In an exemplary embodiment, the body part comprises a finger. In an exemplary embodiment, the body part comprises a hand. In an exemplary embodiment, the body part comprises a forearm. In an exemplary embodiment, the body part comprises a leg. In an exemplary embodiment, the body part comprises a foot. In an exemplary embodiment, the measuring and recording the skin temperature of a body part comprises multiple temperature measurement at different points on the body part.

A computer program for determining one or more health conditions has been described comprising a retrieval engine adapted to retrieve a plurality of temperature data from a database, the temperature data comprising a baseline temperature, a temperature drop from the baseline temperature having a first slope, a lowest temperature achieved, a temperature rise from the lowest temperature achieved having a second slope, a peak temperature, and a stabilization temperature; a processing engine adapted to process data retrieved by the retrieval engine, and a diagnosis engine operable to determine one or more health conditions based upon the retrieved temperature data.

For the present study, sitting blood pressure was recorded in the left arm before DTM testing, using an Omron HEM 705 CP semi-automated sphygmomanometer (Omron Healthcare, Inc., Bannockburn, Ill., USA). Digital thermal measurement (DTM) was carried using a VENDYS 5000BC™ DTM system as disclosed herein in reference to FIG. 1-9 (Endothelix, Inc., Houston, Tex., USA). The device comprises a computer-based thermometry system (0.01° F. thermal resolution) designed and implemented as disclosed herein and including two fingertip thermocouple probes, coupled to a PC. The experimental protocol and data collection are controlled by software implementing the steps of FIG. 3-5. The probes are designed to minimize the area of skin-probe contact, pressure on fingertip, and drift in the baseline temperature. A standard sphygmomanometer cuff and compressor permits controlled occlusion-hyperemia. Subjects fasted overnight and refrained from smoking, alcohol or caffeine ingestion and use of any vasoactive medications on the day of the testing. Subjects remained seated, with the forearms supported at knee level. DTM probes were affixed to the index finger of each hand. After a period of stabilization of basal skin temperature, the right upper arm cuff was rapidly inflated to 200 mmHg for 2 minutes, and then rapidly deflated to invoke reactive hyperemia distally. Temperature was measured in both fingers throughout the protocol, until approximately three minutes after cuff deflation.

It has been observed that in a given individual, if tested on different occasions, may have “intra-individual” variability in measurements of vascular reactivity This is similar to blood pressure variability where is well recognized that measurement of brachial vasoreactivity may show marked variations including diurnal, postprandial, and positional variability. In addition, other variables including for example, ambient temperature and recent exercise or anxiety may influence results. At a given test time, a subject may have a baseline temperature of 35 degrees C, a T_{F} of 2 degrees C. and a T_{R} of 0.5 degrees. A subject like first subject has a baseline temperature which is significantly greater than the ambient temperature, and it is expected that such a subject will experience a higher than normal T_{F} and a lower than normal T_{R}. A subject may have a baseline temperature which relatively high and exceeds the individual's core temperature, and is expected to experience a higher than normal T_{F} and a lower than normal T_{R}. On another occasion the same subject will be found to have a low baseline temperature such as for example 25 degrees C., a T_{F} of 1 degree C. and a T_{R} of 3 degrees. In this second instance the subject has a baseline temperature which is close to the ambient temperature, and it is expected that the subject will experience a lower than normal T_{F} and a higher than normal T_{R}. Furthermore, a subject having a baseline temperature which is close to the subject's core temperature is expected to experience a lower than normal T_{F} and a higher than normal T_{R}. Certain of these variables are controlled by multiple measurements and standardized settings for measurement.

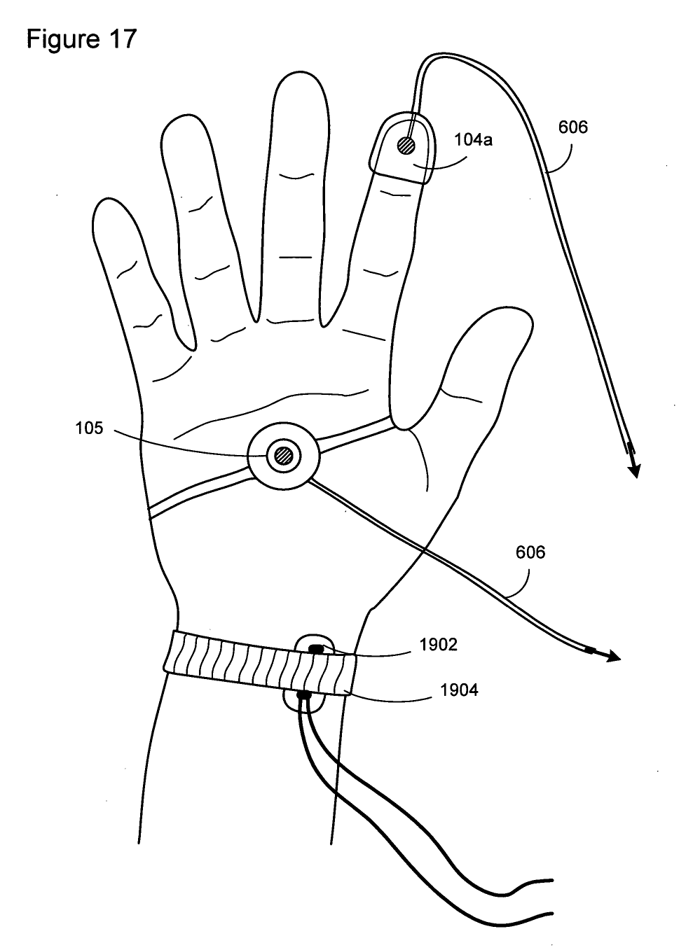

“Cold Fingers” in digital thermal monitoring of vascular reactivity: “Cold finger” is a result of increased sympathetic nervous system activity and can interfere with digital thermal monitoring. Since the fingertip is highly innervated by sympathetic nerves, measuring temperature at the palm and fingertip simultaneously as depicted for example in FIG. 8 or 17 can provide an indicator of sympathetic vasomotor activity and may help in accurate assessment of hyperemia induced vascular reactivity and endothelial function. In other embodiment, temperature is monitored in sites having a reduced neurovascular component, including a finger webbing, a palm and the forearm. Although in the forearm it has been observed that a smaller temperature decrease and rebound is detectable, the ratio is comparable to that shown at the fingertip is observed in persons having normal endothelial function. In individuals for whom the neurovascular response makes finger tip measurement unreliable, forearm temperature is available as a surrogate. A fingertip/forearm ratio could also be used.

To avoid “cold finger” in first place, subjects are typically asked to sit and relax for 5 minutes before fingertip temperature is originally measured. In some individuals this period may need to be prolonged to 20-30 minutes or longer, preferably including a relatively quiet, temperature controlled environment. Where the initial fingertip temperature is lower than 28° C., the group that are required to have a prolonged period of waiting and relaxing to warm up, further warming may include a warming box at constant temperature, electronic lamp (infrared for example), commercial hand warmers, as well as warming in water. Heat, including by washing or immersing hands with warm water, is intended to result in parasympathetic stimulation and relaxation of the arterioles in the fingertip. After 5-15 minutes of immersion in warm water, cold fingers usually warm up and upon reaching stable temperature the digital thermal monitoring can be performed. Where water warming is employed, subsequent evaporative effects should be taken into consideration. An optimum baseline fingertip temperature would be the middle point between room temperature and core body temperature (e.g. ˜31-32° C.).

Other solution for obtaining accurate measurements involves discriminating between neurovascular responses (autonomic response) from hyperemia vascular reactivity responses. Measuring temperature at an anatomic location with maximum sympathetic effect, such as at the fingertip, versus anatomic locations with minimum sympathetic effects, such as on the palm, can help distinguish neurovascular responses from hyperemic vascular responses. A combination of instruments including finger mounted thermal energy sensor 104a and palm mounted temperature sensor 105 depicted in FIG. 17, can be used to distinguish a neurovascular response (autonomic response) from hyperemia vascular reactivity response using thermal monitoring.

In one embodiment of the invention, a mental challenge test is employed to identify a hyperactive sympathetic nervous system and thus to identify those individuals who are prone to develop sustained hypertension. Responses are monitored for an increase in vasoconstriction by looking at increased temperature rather than increased blood pressure. The sympathetic nervous response is assessed for response to stressful tests, i.e. challenging mathematical problems or stressful movies/pictures. Temperature of the fingertip and palm are continuously measured. A determination of the relative hyperactivity of the sympathetic nervous system is based on the behavior of palm and fingertip temperature before, during and after the mental challenge test. This test can be combined with other markers of stress, e.g temperature response along with heart rate or respiratory rate or blood pressure to further evaluate the body's reactivity to stress.

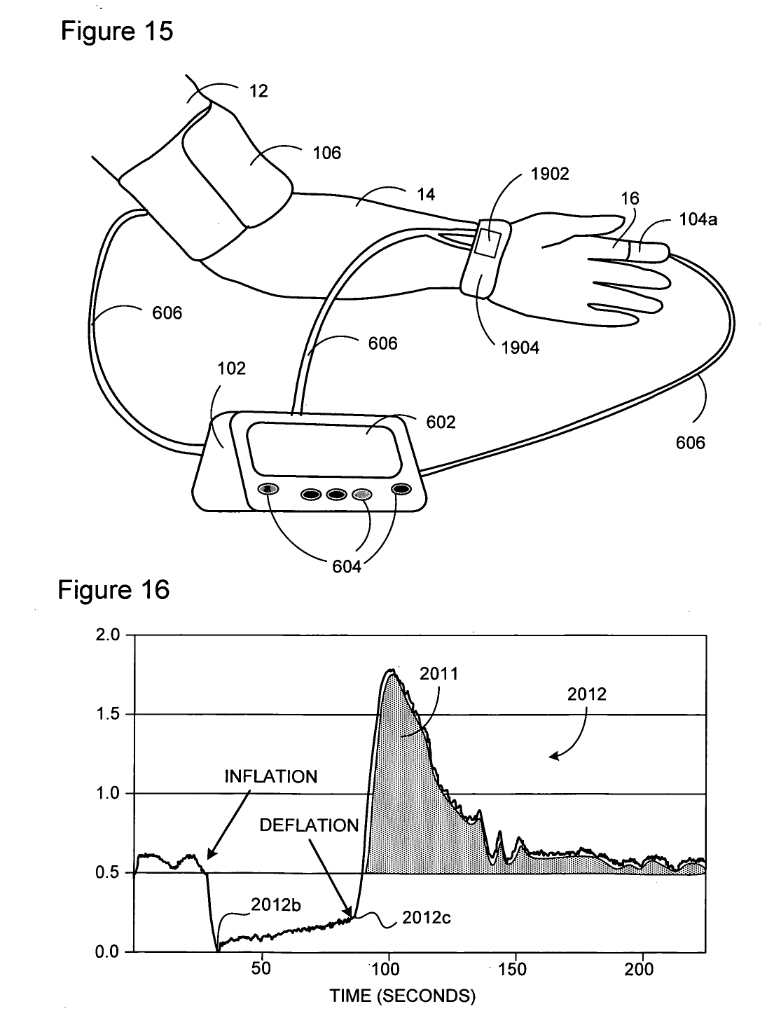

Combined Measures of Vascular Reactivity: Temperature of a digit such as a fingertip in response to vasostimulation represents a fundamental form of vascular reactivity that has contributing components from various sources including endothelial reactivity, smooth muscle reactivity and neurovascular reactivity. Because DTM has a neurovascular component, individuals who persistently exhibit “cold-finger” may be studied by including methods that may be less susceptible to neurovascular influences in the given individual. Thus, in one embodiment, methods and apparatus for comprehensive assessment of vascular function are provided by combining regional and/or digital temperature changes with changes in peak systolic Doppler velocity measurement by Doppler ultrasonography. This combination of thermography and Doppler ultrasonography is herein termed “thermodoppler.” For example, and as depicted in FIG. 15 and FIG. 16, the radial artery can be placed under continuous Doppler measurement together with fingertip or palm thermal monitoring before and after cuff occlusion test. In one embodiment, the probe is bidirectional Doppler probe 1902 which is be placed over the radial artery and held in place by any number of attachments known in the art, including adhesives or, for example, a wrist band 1904. Doppler data as seen in FIG. 16 is obtained by continuous monitoring of peak systolic Doppler velocity decreases after occlusion from its maximum immediately after release of the cuff (cuff deflation) and declining over time to base velocity before occlusion. This response inversely correlates with distal vascular resistance. Immediately after releasing the cuff, resistance is minimum. Upon perfusion the resistance increases back to baseline resistance. The speed of return to baseline resistance, the area 2011 under the produced curve as well as the slope, can be used to study the function of the resistant vasculature. Decreased ability of body to resume resistance or decreased ability of body to drop resistance after occlusion is indicative of inability of the vasculature to respond appropriately to changes in perfusion.

The Doppler pulse velocity curve can be used as a non-invasive correlate of factors such as pH of the hand, calcium ions and metabolic factors affecting the distal microvascular resistance. In summation, the curve can be calibrated to study, non-invasively, factors affecting vascular resistance.

In digital finger temperature studies of vascular function, a somewhat delayed temperature response occurs that may be a result of delayed vasodilation seen in conduit (macro or large) arteries such as the brachial artery. The vasodilation occurs typically after 30 to 60 seconds. However, the Doppler pulse velocity response is maximum immediately after release, and therefore is likely to represent a microvessel response known as the resistant vessel response. Therefore, the combined “thermodoppler” studies of vascular function may provide a more comprehensive assessment of vascular function as result of hyperemia induced vascular reactivity.

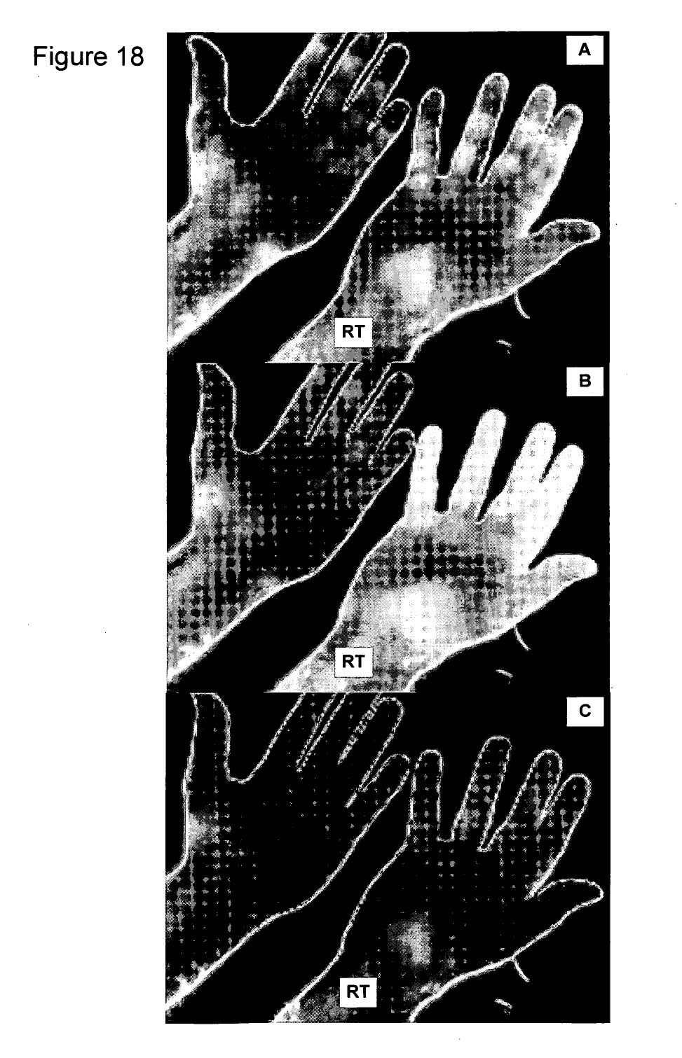

In one embodiment of the invention, infrared imaging is used for thermographic assessment of endothelial dysfunction. Temperatures before, during, and after vasostimulation, such as may be provided by cuff occlusion, are measured by infrared camera. Infrared (IR) thermography is employed to study vascular health before, during, and after a direct vascular stimulant such as nitrate or cuff occlusion. For example, infrared imaging of both hands or feet during cuff occlusion test (before cuff occlusion, during and post occlusion) using infrared thermography results in a comprehensive vascular and neurovascular assessment of vascular response in both hands or feet. FIG. 18 depicts the results of IR thermography of two hands of the same individual where the brachial artery is occluded by an inflated blood pressure cuff on the individual's right arm. In this application, quantitative measurements of temperature changes are generated by numerical analysis of each depth of color in the image. Although black and white images are shown in FIG. 18, the technique may utilize a color infrared camera and thus provide colored images. Alternatively, an image can be created by using multiple thermocouple sensors placed on different parts of the hand. In one embodiment, the multiple sensors 1204a-c are mounted on a glove 1202 as graphically depicted in FIG. 8. If desired, a large number of sensors can be employed, for example 10-20 or more sensors per hand.

In one embodiment, IR thermography is used to assess the condition of a diabetic foot including an assessment of vascular function and reactivity in diabetic patients who are at risk developing foot ulcers or “diabetic foot” as a consequence of vascular disturbances and severely compromised perfusion or ischemia of the foot. Heterogeneity in skin perfusion and vascular health can be seen. The technique can also be used to indicate development of diabetic neuropathy.

Baseline imaging of the feet of a diabetic patient is performed. Imaging is performed after administration of nitrite/nitrate compound e.g. nitrotriglyceride (NTG). Point IR measurement of temperature such as aural thermography can be used for assessment of total body vascular response to vascular stimulant such as nitrate. In such cases a higher temperature response indicates a better vascular function.

In one embodiment, a method and apparatus is provided for using a combination of infrared thermography, digital temperature measurements of vascular reactivity and Doppler ultrasonography simultaneously.

It is understood that variations may be made in the foregoing without departing from the scope of the disclosed embodiments. Furthermore, the elements and teachings of the various illustrative embodiments may be combined in whole or in part some or all of the illustrated embodiments.

Although illustrative embodiments have been shown and described, a wide range of modification, change and substitution is contemplated in the foregoing disclosure and in some instances, some features of the embodiments may be employed without a corresponding use of other features. Accordingly, it is appropriate that the appended claims be construed broadly and in a manner consistent with the scope of the embodiments disclosed herein.

What is claimed is:

1. A method for identifying and controlling neurovascular influence in endothelial function measurement in a patient, comprising:
measuring skin temperatures on a test body part and a contralateral control body part simultaneously;
determining if the skin temperatures are above a set minimum temperature;
if the set minimum temperature is attained, automatically providing a vasostimulant to the subject to substantially cease blood flow to the test body part;
continuously monitoring the skin temperature changes of the test and control body parts during provision of the vasostimulant;
automatically removing the vasostimulant to allow blood flow to the body part;
comparing the skin temperatures to both the test and control body parts before, during and after removal of the vasostimulant; and
determining whether the measured temperature over time of the control body part is substantially stable.

2. The method of claim 1, wherein the patient is stabilized for a period of between 5 and 30 minutes prior to beginning measurement of skin temperatures.

3. The method of claim 1, wherein the set minimum temperature is about 28° C.

4. The method of claim 1, wherein the contralateral body part comprises a plurality of contralateral body parts.

5. The method of claim 1, wherein the body part is a first hand on the subject, and the contralateral body part is a second hand on the subject.

6. The method of claim 1, wherein the body part is a first foot on the subject, and the contralateral body part is a second foot on the subject.

7. The method of claim 1, wherein the body part is a finger on the subject, and the contralateral body part is a corresponding finger on the subject.

8. The method of claim 1, further comprising determining a Doppler flow in the test and/or control body parts.

9. The method of claim 1, further comprising determining a blood flow rate by near infrared spectroscopy.

10. The method of claim 1, wherein the temperature response to the vasostimulant is analyzed to screen the patient for white coat hypertension.

11. The method of claim 1, wherein the temperature response to the vasostimulant is analyzed to monitor the patient's response to mental stress.

12. The method of claim 1, further comprising changing the skin temperature of the body part by heating and/or cooling the body part with a thermal device.

13. The method of claim 1, wherein the temperature response to the vasostimulant is analyzed to determining whether the patient has diabetic foot.

14. The method of claim 1, wherein the temperature response to the vasostimulant is analyzed to determining a status and progression of diabetic foot in the patient.

15. The method of claim 1, wherein the temperature response to the vasostimulant is analyzed to determining a response of the patient to diabetic therapies.

16. The method of claim 1, wherein the steps are implemented by a computer program controlling a thermal energy senor engine, a vasostimulant engine, and a plotting engine.

17. The method of claim 1, wherein the temperature is monitored body parts having a reduced neurovascular component selected from a finger webbing, a palm and a forearm.

18. A method for identifying a status of diabetic foot in a patient, comprising:
determining a measure of perfusion on both feet of a diabetic patient before during and after providing a vasostimulant to the patient;
comparing the measures of perfusion between the feet.

19. The method of claim 18, wherein a baseline measure of perfusion of both feet of the patient is performed and a further measure of perfusion is performed after administration of nitrite/nitrate compound.

20. The method of claim 18, wherein the measure of perfusion is selected from the group consisting of: digital temperature monitoring, Doppler ultrasonography, infrared thermography, and combinations thereof.
